(19) United States
(12) Patent Application Publication
(10) Pub. No.: US 2002/0025983 A1
(43) Pub. Date: Feb. 28, 2002

(54) Vitamin K and essential fatty acids

(76) Inventor: David Frederick Horrobin, Stirling (GB)

Correspondence Address: JACOBSON HOLMAN, PROFESSIONAL LIMITED LIABILITY COMPANY, 400 SEVENTH STREET, N.W., WASHINGTON, DC, 20004, US

(21) Appl. No.: 09/898,458

(22) Filed: Jul. 5, 2001

(30) Foreign Application Priority Data
Jul. 4, 2000 (GB) 0016452.5

(51) Int. Cl. A61K 31/202; A61K 31/122

(52) U.S. Cl. 514/560; 514/682

(57) ABSTRACT

Nutritional and pharmaceutical formulations comprising in combination vitamin K and a source of at least one essential fatty acid (EFA) as the essential ingredients, and foodstuff containing EFA(s) and an artificially elevated quantity of vitamin K are provided for the treatment or prevention of a variety of diseases or conditions.

Vitamin K is a general name for a group of compounds all with similar biological activity. They all contain the 2-methyl-1,4-naphthoquinone nucleus with a lipophilic side chain at position 3. The three best known members are phylloquinone (vitamin K1) which is the only type of vitamin K found in plants. Vitamin K2, the menaquinones, consists of a family of compounds with variable length isoprenyl side chains. Vitamin K3, menadione, is a pro-vitamin which can be converted to vitamin K2 by animals. Menadione and the menaquinones may occasionally have toxic effects in high doses whereas phylloquinone seems to be safe even in massive overdose. Phylloquinone is therefore the preferred form of the vitamin for human use.

Vitamin K compounds are widely distributed in foods. Among animal foods, eggs, butter and liver are good sources and contain amounts of from about 2 to about 50 μg/100 g of the food. Green vegetables are also good sources and may contain from 30 to as much as 800 μg/100 g of the food. Spinach, kale, sprouts and broccoli are good sources. Vegetable oils, and products made from vegetable oils such as margarines and salad dressings, can also be good sources, containing from 10 to 300 μg of vitamin K per 100 g of oil. Olive oil and soy oil are particularly rich in vitamin K. Some vitamin K is also made from gut bacteria although this is difficult to quantitate and may be very variable.

The US Recommended Daily Allowance (RDA) for vitamin K starts at 10 μg/day for infants and rises to 65 μg/day in women and 80 μg/day in men. There is, however, evidence that vitamin K from some foods may be relatively poorly absorbed and there have been suggestions that these RDAs for ordinary foods may be somewhat low (BLMG Gijsbers et al, Effect of food composition on vitamin K absorption in human volunteers, Br J Nutrition 1996; 76:223-229).

The best known role for vitamin K in humans is as a co-factor for the synthesis of six of the proteins involved in blood clotting. These proteins are inactive proenzymes which are converted to active enzymes in the presence of calcium during the coagulation process. These proteins contain an unusual amino acid, gamma-carboxy-glutamate. This is formed by the carboxylation of glutamic acid residues in the protein by the enzyme gamma-glutamyl carboxylase, in a vitamin-K dependent reaction. In the absence of vitamin K, the normal forms of the clotting factors cannot be synthesised. Proteins containing gamma-carboxy-glutamate have become known by the general name of Gla proteins.

For some time it was thought that Gla proteins were confined to the clotting system and it was largely on this basis that the RDAs were estimated. However, it is now known that enzymes with gamma-glutamyl-carboxylase activity are widely distributed in many different tissues and so it is probable that there are many functions of Gla proteins to be discovered. These proteins are particularly abundant in kidney and in bone and so it is assumed that they have particular roles to play in these organs. Two Gla proteins are particularly abundant in bone. Bone Gla protein (BGP, commonly known as osteocalcin) contains three Gla residues and is present in great abundance in bone, dentin and cartilage. Matrix Gla protein (MGP) is also found in substantial amounts in bone, dentin and cartilage. Much ongoing research is trying to identify the roles of these proteins which seem to be involved in determining the strength and resilience of the structure. The kidney Gla proteins may be involved in regulation of calcium excretion so that vitamin K may play a role in integrating the various mechanisms involved in maintaining bone strength (N C Binkley and J W Suttie, Vitamin K nutrition and osteoporosis, J Nutr 1995; 125:1812-21 and C Vermeer et al, Effects of vitamin K on bone mass and bone metabolism, J Nutr 1996; 126:1187S-1191S).

Recently there is evidence that vitamin K can have clinically relevant effects on bone. In women with osteoporosis, a controlled study showed that 45 mg/day of vitamin K2 could reduce the risk of bone fractures and slow down but not prevent a progressive loss of bone mineral density (M Shiraki et al, J Bone Mineral Res 2000; 15:515-521). In a prospective study of 72,000 nurses, women with the lowest quintile of vitamin K intake (109 μg/day and below) had an increased risk of fractures D Feskanich et al, Vitamin K intake and hip fractures in women: a prospective study, Am J Clin Nutr 1999; 69:74-79).

In an older group of men and women, mostly over 70, there was a progressively reducing risk of osteoporotic fracture as vitamin K intake increased.

The lowest risk was in the highest quartile of vitamin K intake of more than 262 μg/day in women and more than 234 μg/day in men (SL Booth et al, Dietary vitamin K intakes are associated with hip fracture but not with bone mineral density in elderly men and women, Am J Clin Nutr 2000; 71:1201-8). The progressive effect of increasing daily intakes suggests that doses of vitamin K much higher than the RDAs may be important in bone health, especially in older people.

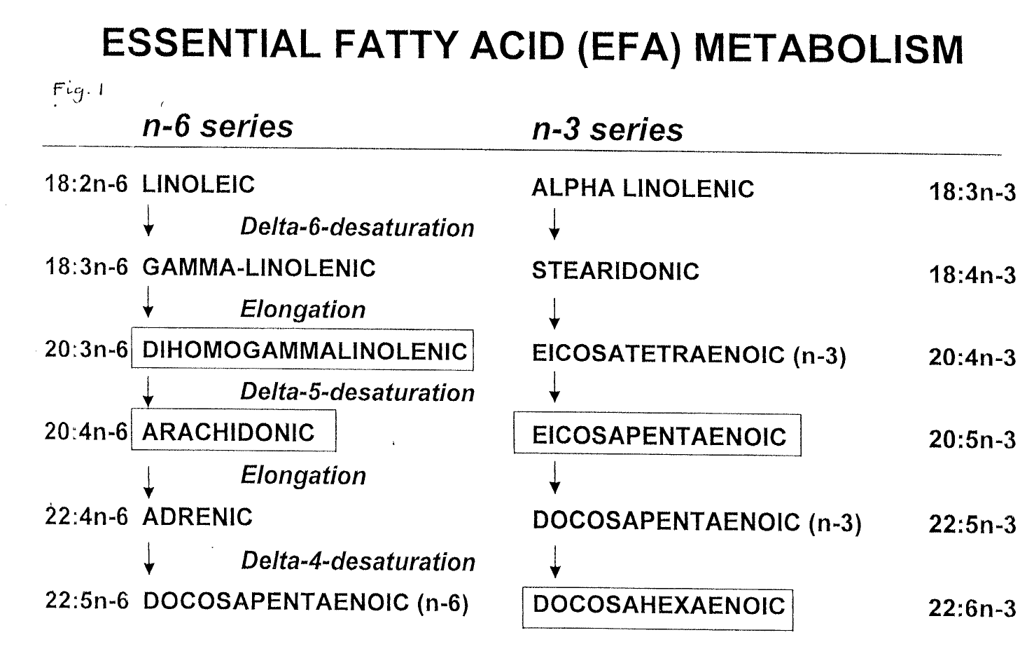

The essential fatty acids (EFAs) are a completely different group of nutrients. There are two types, the omega-6 derived from the parent linoleic acid, and the omega-3 derived from the parent alphalinolenic acid (FIG. 1). The EFAs cannot be synthesised de novo by humans or other mammals. Nor can mammals convert omega-3 EFAs into omega-6 EFAs or vice versa. Mammals can interconvert one omega-6 EFA into another omega-6 EFA via the pathways shown in FIG. 1. Similarly, omega-3 EFAs can be converted one to another. The pathways shown in FIG. 1 usually progress from linoleic acid or alpha-linolenic acid downwards, but retro-conversions are possible.

The EFAs are essential components of complex lipids such as triglycerides, cholesterol esters and phospholipids, and are absolutely required for the normal structure and function of all cell and other membranes in the body. Deficiencies of EFAs cause widespread defects in all body systems. While dietary deficiencies of the parent EFAs, linoleic and alpha-linolenic acids are relatively rare, deficiencies of their metabolites are relatively common because of impaired conversion mechanisms. As a result low levels of fatty acids like dihomogammalinolenic acid (DGLA), arachidonic acid (AA) and adrenic acid (AdrA) of the n-6 series and of stearidonic acid (SA), eicosapentaenoic acid (EPA), docosapentaenoic acid (DPA) and docosahexaenoic acid (DHA) of the n-3 series have been commonly reported. Such low levels have been found in skin diseases including atopic eczema; reproductive system disorders including premenstrual syndrome, breast pain and menstrual pain; diabetes; cardiovascular disorders; bone disorders; kidney diseases; psychiatric diseases including schizophrenia, depression, stress and attention deficit hyperactivity disorder; and many other conditions. Treatment with EFAS, especially with gamma-linolenic acid (GLA) of the n-6 series and with EPA and DHA of the n-3 series has been reported to be associated with a wide range of beneficial effects. These effects have been reported to be enhanced by certain nutrients such as zinc and vitamin B6 which are important in EFA metabolism.

The present invention is based on the inventor's finding of a completely unexpected and hitherto unreported interaction between vitamin K and EFAs.

The present invention provides nutritional and pharmaceutical formulations comprising in combination a source of vitamin K and a source of at least one essential fatty acid (EFA), in which the concentration of vitamin K is not less than 1000 μg/100 g. Preferably, the concentration of vitamin K is not less than 1000 μg/10 g. The formulations of the invention preferably comprise between 50 μg and 100 mg vitamin K and between 50 mg and 100 g of the EFA. These are to provide a daily dose of these amounts and the formulation may be in the form of a single dosage, to provide these intakes in one go, or in the form of divided doses.

The present invention further provides nutritional and pharmaceutical formulations comprising in combination a source of Vitamin K and a source of at least one EFA, but which exclude proteins or amino acids as part of the active ingredients of the formulations.

Vitamin K is preferably in the form of phylloquinone (vitamin K1).

The EFA may be selected from the n-6 series: gamma-linolenic acid, dihomogammalinolenic acid, arachidonic acid and adrenic acid, and combinations of these EFAs. Alternatively, the EFA is selected from the n-3 series: stearidonic acid, eicosapentaenoic acid, docosapentaenoic acid and docosahexaenoic acid, and combinations of these EFAs. In a further embodiment of the present invention at least one n-6 EFA is present with at least one n-3 EFA, each EFA selected from the above lists.

The active ingredient of the nutritional or pharmaceutical composition may consist essentially wholly of EFA and vitamin K or, alternatively, the formulations of the present invention may further comprise one or more essential vitamins and/or minerals or one or more pharmaceutical drugs.

The present invention further provides foodstuff which already contain EFAs to which have been added vitamin K in an amount to raise the vitamin K content of the food to 1000 μg/100 g food, or more. The specific EFA(s) content may also be raised artificially by the addition of one of more EFAs.

It is desirable that the present invention still further provides foodstuffs containing EFAS but excluding proteins or amino acids as part of the active ingredients. Added to the composition is vitamin K to artificially raise the vitamin K content of the foodstuff. The specific EFA(s) content may also be raised artificially by the addition of one of more EFAs.

The present invention still further provides foodstuff which naturally contains clinically or nutritionally small amounts of vitamin K and/or EFA(s) but to which has been added vitamin K and EFAs, for example to the dosage regime of the formulations of the first aspect of the present invention. Milk and other dairy products or simulated dairy products are particularly appropriate for this type of enrichment.

The foodstuffs and nutritional or pharmaceutical formulations of the present invention may be used to treat or prevent a variety of diseases or conditions. These may include:

premenstrual or menstrual disorders of any kind;

bone or calcium disorders of any kind, including osteoporosis;

metabolic or cardiovascular disorders including diabetes, obesity, elevated blood cholesterol or triglyceride levels or cardiovascular disorders;

stress, mental, psychological, psychiatric or neurological disorders;

skin disorders;

asthma or other respiratory disorder;

arthritis or any form of inflammatory, gastrointestinal, kidney or reproductive system disorder.

The present invention further provides a method of treatment or prevention of diseases or conditions, including those mentioned above, by the administration of a combination of vitamin K and an EFA, preferably at the dosage rate of between 50 μg and 100 mg vitamin K and between 50 mg and 100 g EPA. In particular, the disorders to be treated are skin disorders and premenstrual or menstrual conditions. Bone disorders are also of particular importance.

The vitamin K may be provided in any form which has biological vitamin K activity in mammals. However, because of its safety and known activity, vitamin K1 (phylloquinone) is the preferred form. The formulations may provide for an increase in vitamin K intake in a nutritional or pharmaceutical formulation or food of from 50 μg to 100 mg per day. At the same time the formulations should provide for an increase in the intake of one or more of the desired EFAs of between 50 mg and 50 g per day. Depending on the problem to be addressed, any of the EFAs shown in FIG. 1 may be used. Linoleic acid, alpha-linolenic acid, GLA, DGLA, AA, AdrA, SA, EPA, DPA and DHA are likely to be preferred ingredients for particular purposes. The EFAs may be provided as purified or partially purified compounds or may be supplied by natural oils which are rich in one or more EFAs. For example, borage and evening primrose oils are good sources of GLA, Echium oils are good sources of SA, marine oils are often good sources of EPA, DPA, DHA and sometimes AA, while oils from various microbial sources, including fungal and algal oils can be sources of GLA, DGLA, AA, SA, EPA or DHA. The EFAs can be in any chemical form which is absorbed into the body and incorporated into body lipids. Such forms include but are not limited to free acids, sodium, potassium, lithium and other salts, triglycerides and other glycerides, cholesterol, ethyl, methyl and other esters, amides, and phospholipids.

The vitamin K and the EFA when used for pharmaceuticals or nutritional supplements can be incorporated into any appropriate dosage form known to those skilled in the art. Such dosage forms include soft and hard gelatin capsules, tablets, microcapsules of various types, powders and carriers of various types, liquids, emulsions, micelles and any other forms. Flavourants, colourants, emulsifiers and conventional diluents and excipients may be included, alone or in combination. Examples of formulations of the dosage follow.

### EXAMPLE 1

500 mg or 100 mg hard or soft gelatin capsules in which a natural oil containing GLA is formulated with vitamin K, preferably phylloquinone, at a level between 0.05 and 1.0 mg per capsule.

### EXAMPLE 2

500 mg or 1000 mg hard or soft gelatin capsules where the natural oil contains stearidonic acid (SA), eicosapentaenoic acid (EPA), docosapentaenoic acid (DPA), docosahexaenoic acid (DHA), dihomogammalinolenic acid (DGLA) or arachidonic acid (AA).

### EXAMPLE 3

500 mg or 1000 mg hard or soft gelatin capsules containing gamma-linolenic acid (GLA) in either triglyceride or ethyl-ester form in which the purity of the GLA is greater than 50% and preferably greater than 90% and in which vitamin K, preferably phylloquinone, is provided at a level of between 0.05 and 5.0 mg/capsule.

### EXAMPLE 4

500 mg or 1000 mg hard or soft gelatin capsules containing SA, EPA, DPA, DHA, DGLA or AA or a mixture of these fatty acids and in which vitamin K, preferably phylloquinone, is provided at a level of between 0.05 and 5.0 mg/capsule.

### Example 5

Liquid natural oils containing:

GLA; or

SA, EPA, DPA, DHA, DGLA or AA; or

GLA in triglyceride or ethyl-ester form in which the purity of the GLA is greater than 50% and preferably greater than 90%;

or to which are added 1000 micrograms/100 g and 100 mg/100 g of vitamin K, preferably in the phylloquinone form. Such oils may be used themselves, or flavoured using appropriate flavourings, or incorporated into microcapsules made from any appropriate material or added to foodstuffs of any appropriate type.

### EXAMPLE 6

Milk or milk products from any edible source, either animal or vegetable, such as soy milk, to which are added phylloquinone to raise the concentration to over 1000 microg/100 g and preferably to over 5000 microg/100 g together with one or more fatty acids selected from GLA, DGLA, AA, SA, EPA, DPA and DHA, to raise the concentration of each selected fatty acid to more than 100 mg/100 g and preferably to more than 1000 mg/100 g.

When used in foods, the formulations may be prepared by increasing the concentration of vitamin K in the food to 1000 μg/100 g or more. With some foods, such as milks, dairy products or vegetable oils, moderate amounts of EFAs may already be present in the natural food. Increasing the vitamin K content of such foods to a level above that present in any natural food is within the framework of the invention.

Alternatively, in addition to raising the vitamin K content of an EFA-containing food to over 1000 μg/100 g, the desired EFA may also be added to the food to raise the amount provided. Natural and soy or other vegetable-based milks, soy and related products, dairy products including yogurts, cheeses, butters, margarines, or any other types of foods may all be treated in this way to provide a combination of vitamin K and an EFA.

These formulations may be used for general health purposes, or for specific conditions where either EFAs or both have been found to be helpful. These conditions include premenstrual and menstrual disorders, skin disorders, diabetes, elevated is cholesterol and triglyceride levels, cardiovascular disorders, arthritis and any form of inflammatory disorder, respiratory disorders such as asthma, gastro-intestinal, urinary and reproductive system disorders in both sexes, mental, psychological and psychiatric disorders such as stress, chronic fatigue, behavioural problems in children and adults, depression, alcoholism and more serious psychiatric disorders such as schizophrenia and bipolar disorder, neurological disorders such as Parkinsonism, and any form of dementia and any other form of illness in which the combinations are found to be helpful. Osteoporosis and related disorders of bone and calcium metabolism are likely to provide particularly important uses for the invention.

### BRIEF DESCRIPTION OF THE FIG.S

FIG. 1 The n-6 and n-3 essential fatty acids

### EXPERIMENTAL DATA

A woman with atopic dermatitis and with mild premenstrual syndrome was recommended to take 3 g/day of evening primrose oil (EPO). EPO is a widely used nutritional supplement for these problems. It contains about 70% of linoleic acid, but more importantly contains 8-12% of GLA which can by-pass a block in the conversion of linoleic acid to GLA which can occur in many situations, including atopic dermatitis, stress and menstrual disorders. Not everyone responds to EPO but this is an exceptionally safe nutritional supplement and does not cause any important side effects. However, to my surprise in this woman the EPO not only failed to have any therapeutic effect but caused a range of unusual side effects including facial reddening and rashes, a worsening of her dermatitis, gastrointestinal disturbances and anxiety and depression. As a result of a series of investigations, she was found to have a vitamin K deficiency, possibly partly due to dietary problems and partly due to gastrointestinal infections which had necessitated the use of antibiotics which had probably changed her gut bacteria. The vitamin K deficiency was corrected by vitamin K1 supplements but this did not improve her skin or her premenstrual syndrome. As an experiment she was then cautiously given EPO again. This time there were no adverse effects at all, her skin improved and her premenstrual syndrome was resolved. This case suggested a hitherto unsuspected and important positive interaction between vitamin K and EFAs.

A second woman presented with severe menstrual cramps and mild premenstrual syndrome. I suggested that she should take a low dose of EPO (1 g/day) to help with her premenstrual syndrome and a higher dose (4 g/day) of a fish oil containing 23% of EPA and 8% of DHA to help with her menstrual cramps. Unfortunately she showed no response in either of her problems. She had a normal diet and no evidence of vitamin K deficiency but I wondered whether giving vitamin K might help. She therefore took 2 mg (2000 μg) per day of a vitamin K1 supplement for a month which also had no effect on her menstrual problems. However, on reintroducing the EPO and fish oil, her premenstrual syndrome disappeared completely and her menstrual pain was greatly reduced.

These cases show that vitamin K can greatly improve the therapeutic effects of EFAs, reducing any side effects and enhancing therapeutic effects.

1. Nutritional and pharmaceutical formulations comprising in combination a source of vitamin K and a source of at least one essential fatty acid (EFA), in which the concentration of vitamin K is not less than 1000 μg/100 g.

2. Nutritional and pharmaceutical formulations according to claim 1, in which the concentration of vitamin K is not less than 1000 μg/10 g.

3. Nutritional and pharmaceutical formulations according to claim 1 which provide a daily dose between 50 μg and 100 mg vitamin K and between 50 mg and 100 g of the EFA.

4. Nutritional and pharmaceutical formulations according to claim 1 in which the form of vitamin K used is phylloquinone (vitamin K1).

5. Nutritional and pharmaceutical formulations according to claim 1 in which the EFA is selected from gamma-linolenic acid, dihomogammalinolenic acid, arachidonic acid and adrenic acid, and combinations of these EFAs.

6. Nutritional and pharmaceutical formulations according to claim 1 in which the EFA is selected from stearidonic acid, eicosapentaenoic acid, docosapentaenoic acid and docosahexenoic acid, and combinations of these EFAS.

7. Nutritional and pharmaceutical formulations according to claim 1 in which there is at least one n-6 EFA and at least one n-3 EFA present, the n-6 EFA(s) selected from gamma-linolenic acid, dihomogammalinolenic acid, arachidonic acid and adrenic acid, and combinations of these acids, and the n-3 EFA(s) selected from stearidonic acid, eicosapentaenoic acid, docosapentaenoic acid and docosahexaenoic acid, and combinations of these acids.

8. Nutritional and pharmaceutical formulations according to claim 1 in which the active ingredient consists essentially wholly of EFA and vitamin K.

9. Nutritional and pharmaceutical formulations according to claim 1 further comprising one or more essential vitamins and/or minerals or one or more pharmaceutical drugs.

10. Nutritional and pharmaceutical formulations comprising in combination a source of vitamin K and a source of at least one essential fatty acid (EFA), in which proteins and amino acids are absent from the active ingredients of the formulation.

11. Nutritional and pharmaceutical formulations according to claim 10 which provide a daily dose between 50 μg and 100 mg vitamin K and between 50 mg and 100 g of the EFA.

12. Nutritional and pharmaceutical formulations according to claim 10 in which the form of vitamin K used is phylloquinone (vitamin K1).

13. Nutritional and pharmaceutical formulations according to claim 10 in which the EFA is selected from gamma-linolenic acid, dihomogammalinolenic acid, arachidonic acid and adrenic acid, and combinations of these EFAs.

14. Nutritional and pharmaceutical formulations according to claim 10 in which the EFA is selected from stearidonic acid, eicosapentaenoic acid, docosapentaenoic acid and docosahexenoic acid, and combinations of these EFAs.

15. Nutritional and pharmaceutical formulations according to claim 10 in which there is at least one n-6 EFA and at least one n-3 EFA present, the n-6 EFA(s) selected from gamma-linolenic acid, dihomogammalinolenic acid, arachidonic acid and adrenic acid, and combinations of these acids, and the n-3 EFA(s) selected from stearidonic acid, eicosapentaenoic acid, docosapentaenoic acid and docosahexaenoic acid, and combinations of these acids.

16. Nutritional and pharmaceutical formulations according to claim 10 in which the active ingredient consists essentially wholly of EFA and vitamin K.

17. Nutritional and pharmaceutical formulations according to claim 10 further comprising one or more essential vitamins and/or minerals or one or more pharmaceutical drugs.

18. Foodstuff which already contain EFAs to which have been added vitamin K in an amount to raise the vitamin K content of the food to 1000 μg/100 g food, or more.

19. Foodstuff which already contain EFAs to which have been added vitamin K in an amount to raise the vitamin K content of the food to 1000 μg/10 g food, or more.

20. Foodstuff according to claim 18 in which the specific EFA(s) content has been raised by the addition of one of more EFAs.

21. Foodstuff which naturally contains clinically or nutritionally small amounts of vitamin K and/or EFA(s) to which has been added vitamin K and EFAs.

22. A method of treating or preventing a variety of diseases or conditions including:
premenstrual or menstrual disorders of any kind;
bone or calcium disorders of any kind, including osteoporosis;
metabolic or cardiovascular disorders including diabetes, obesity, elevated blood cholesterol or triglyceride levels or cardiovascular disorders;
stress, mental, psychological, psychiatric or neurological disorders;
skin disorders;
asthma or other respiratory disorder;
arthritis or any form of inflammatory, gastrointestinal, kidney or reproductive system disorder;
using nutritional and pharmaceutical formulations comprising in combination a source of vitamin K and a source of at least one essential fatty acid (EFA), in which the concentration of vitamin K is not less than 1000 μg/100 g.

23. The method according to claim 22 in which the concentration of vitamin K is not less than 1000 μg/10 g.

24. The method according to claim 22 which provides a daily dose between 50 μg and 100 mg vitamin K and between 50 mg and 100 g of the EFA.

25. The method according to claim 22 in which the form of vitami n K used is phylloquinone (vitamin K1).

26. The method according to claim 22 in which the EFA is selected from gamma-linolenic acid, dihomogammalinolenic acid, arachidonic acid and adrenic acid, and combinations of these EFAs.

27. The method according to claim 22 in which the EFA is selected from stearidonic acid, eicosapentaenoic acid, docosapentaenoic acid and docosahexenoic acid, and combinations of these EFAs.

28. The method according to claim 22 in which there is at least one n-6 EFA and at least one n-3 EFA present, the n-6 EFA(s) selected from gamma-linolenic acid, dihomogammalinolenic acid, arachidonic acid and adrenic acid, and combinations of these acids, and the n-3 EFA(s) selected from stearidonic acid, eicosapentaenoic acid, docosapentaenoic acid and docosahexaenoic acid, and combinations of these acids.

29. The method according to claim 22 in which the active ingredient consists essentially wholly of EFA and vitamin K.

30. The method according to claim 22 further comprising one or more essential vitamins and/or minerals or one or more pharmaceutical drugs.

31. A method of treating or preventing a variety of diseases or conditions including:
premenstrual or menstrual disorders of any kind;
bone or calcium disorders of any kind, including osteoporosis;
metabolic or cardiovascular disorders including diabetes, obesity, elevated blood cholesterol or triglyceride levels or cardiovascular disorders;
stress, mental, psychological, psychiatric or neurological disorders;
skin disorders;
asthma or other respiratory disorder;
arthritis or any form of inflammatory, gastrointestinal, kidney or reproductive system disorder;
using nutritional and pharmaceutical formulations comprising in combination a source of vitamin K and a source of at least one essential fatty acid (EFA), in which proteins and amino acids are absent from the active ingredients of the formulation.

32. The method according to claim 31 which provides a daily dose between 50 μg and 100 mg vitamin K and between 50 mg and 100 g of the EFA.

33. The method according to claim 31 in which the form of vitamin K used is phylloquinone (vitamin K1).

34. The method according to claim 31 in which the EFA is selected from gamma-linolenic acid, dihomogammalinolenic acid, arachidonic acid and adrenic acid, and combinations of these EFAs.

35. The method according to claim 31 in which the EFA is selected from stearidonic acid, eicosapentaenoic acid, docosapentaenoic acid and docosahexenoic acid, and combinations of these EFAs.

36. The method according to claim 31 in which there is at least one n-6 EFA and at least one n-3 EFA present, the n-6 EFA(s) selected from gamma-linolenic acid, dihomogammalinolenic acid, arachidonic acid and adrenic acid, and combinations of these acids, and the n-3 EFA(s) selected from stearidonic acid, eicosapentaenoic acid, docosapentaenoic acid and docosahexaenoic acid, and combinations of these acids.

37. The method according to claim 31 in which the active ingredient consists essentially wholly of EFA and vitamin K.

38. The method according to claim 31 further comprising one or more essential vitamins and/or minerals or one or more pharmaceutical drugs.

39. A method of treating or preventing a variety of diseases or conditions including:
premenstrual or menstrual disorders of any kind;
bone or calcium disorders of any kind, including osteoporosis;
metabolic or cardiovascular disorders including diabetes, obesity, elevated blood cholesterol or triglyceride levels or cardiovascular disorders;
stress, mental, psychological, psychiatric or neurological disorders;
skin disorders;
asthma or other respiratory disorder;
arthritis or any form of inflammatory, gastrointestinal, kidney or reproductive system disorder;
using foodstuff which already contain EFAs to which have been added vitamin K in an amount to raise the vitamin K content of the food to 1000 μg/100 g food, or more.

40. The method according to claim 39 using foodstuff which already contain EFAs to which have been added vitamin K in an amount to raise the vitamin K content of the food to 1000 μg/10 g food, or more.

41. The method according to claim 39 in which the specific EFA(s) content has been raised by the addition of one of more EFAs.

42. The method according to claim 39 using foodstuff which naturally contains clinically or nutritionally small amounts of vitamin K and/or EFA(s) to which has been added vitamin K and EFAs.
